(19) United States
(12) Patent Application Publication
(10) Pub. No.: US 2006/0198832 A1
(43) Pub. Date: Sep. 7, 2006

(54) Peptide drugs for chronic lymphocytic leukemia (CLL) and other cancers

(76) Inventors: Arnold Satterthwait, San Diego, CA (US); Xiuwen Zhu, San Diego, CA (US); Shinichi Kitada, San Diego, CA (US); John Reed, Rancho Santa Fe, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/267,828

(22) Filed: Nov. 3, 2005

Related U.S. Application Data
(60) Provisional application No. 60/624,947, filed on Nov. 3, 2004.

(51) Int. Cl. A61K 38/54; C12N 9/99

(52) U.S. Cl. 424/94.2; 435/184

(57) ABSTRACT

The present invention provides a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1). Further provided herein is a composition containing a delivery agent and a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1).

This application claims the benefit of priority of provisional application Ser. No. 60/624,947, filed on Nov. 3, 2004, which is incorporated herein by reference.

This invention was made with government support under CA815334 and CA095338 awarded by the National Institutes of Health. The government has certain rights in this invention.

### BACKGROUND OF THE INVENTION

1. Field of the Invention

The present invention relates generally to the fields of cancer and molecular medicine and, more specifically, to apoptosis-inducing peptides and peptidomimetics for treatment of leukemias and other cancers.

### BACKGROUND INFORMATION

The bcl-2 gene family encodes a divergent group of proteins that regulate programmed cell death and stress-induced apoptosis by an evolutionarily conserved mechanism found in both humans and worms. One subgroup of the Bcl-2 family proteins, including mammalian Bcl-2, is required for cell survival (Adams and Cory, Science 281:1322-1326 (1998); and Hengartner, Nature 407:770-776 (2000)) In contrast, two other subgroups, the “Bax/Bak-like proteins” (Gross et al., Genes Dev. 13:1899-1911 (1999)) and the more distantly related “BH3-only proteins” (Huang and Strasser, Cell 88:347-354 (2000)) are both required for cell death. During embryogenesis, members of the Bcl-2 family cooperate to control developmentally programmed cell death (Jacobson et al., Cell 88:347-354 (2000)). After birth, members of the Bcl-2 family play critical roles in regulating programmed cell death in the hematopoietic system and in tissue homeostasis and mammary gland involution (Jacobson et al., supra, 2000; Newton and Strasser, Adv. Immunol. 76:179-226 (2001)). In addition to regulating cell death triggered by developmental and physiological cues, members of the Bcl-2 protein family also control apoptosis induced by cytotoxic stresses such as anti-cancer drugs (Adams and Cory, supra, 1998).

Abnormalities in cell death control can cause or contribute to a variety of diseases including cancer. As an example, overexpression of the apoptosis inhibitor Bcl-2 can promote tumorigenesis (Vaux et al., Nature 335:440-442 (1988); and Strasser et al., Nature 348:331-333 (1990)). Bcl-2 can also play a role in the efficacy of anti-cancer therapies. Whereas radiation and the majority of chemotherapeutic drugs trigger apoptosis in susceptible cells including tumor-derived cells and normal cells, Studies with normal lymphocytes and lymphomas clearly demonstrate that Bcl-2 overexpression inhibits radiation and anti-cancer drug-induced apoptosis in short term assays and promotes long-term survival and growth (Strasser et al., Cell 79:329-339 (1994); and Schmitt et al., Nat. Med. 6:1029-1035 (2000)). Essentially, expression of the pro-survival protein Bcl-2 renders cancer cells refractory to effective treatment (Coultas and Strasser, Sem. Cancer Biol. 13:115-123 (2003)).

Current cancer therapies which aim, at least in part, to reduce Bcl-2 expression or activity have only been partially successful. Thus, there is a need for novel molecules which counteract Bcl-2 activity and render cancer cells sensitive to treatment by apoptosis-inducing agents. The present invention provides this need and provides related advantages as well.

### SUMMARY OF INVENTION

The present invention provides a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1).

The present invention also provides a composition containing a delivery agent and a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1).

Further provided herein are methods of treating leukemia in a patient by administering to the patient a composition which contains a delivery agent and a modified BAD peptide or peptidomimetic that includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1).

### BRIEF DESCRIPTION OF THE DRAWINGS

FIG. 1 shows a competition ELISA for binding of wild type and modified BAD peptides to Bcl-2.

FIG. 2 shows that cell viability of chronic lymphocytic leukemia (CLL) cells is significantly reduced in cells electroporated with the modified BAD 2Ser/Ala peptide SEQ ID NO: 2 as compared to wild type BAD peptide SEQ ID NO: 1 and control BAD peptide SEQ ID NO: 4.

FIG. 3 shows the reduced cell viability of patient TJK379 chronic CLL cells treated with a modified BAD 2Ser/Ala peptide containing a polyarginine cell penetration sequence (SEQ ID NO: 6). Viability of cells treated with a wild type BAD peptide containing a polyarginine cell penetration sequence was negligibly effected.

FIG. 4 shows the reduced cell viability of patient TJK197 chronic CLL cells treated with a modified BAD 2Ser/Ala peptide containing a polyarginine cell penetration sequence (SEQ ID NO: 6) as compared to wild type and control BAD peptides SEQ ID NOS: 28 and 29.

FIG. 5 shows the effect of a modified BAD peptide on Bcl-2 levels in chronic lymphocytic leukemia cells incubated with the indicated peptides for four hours. Left panel: TJK160 CLL patient cells. Right panel: TJK357 CLL patient cells. SEQ ID NO: 6 is a modified BAD 2Ser/Ala peptide containing a polyarginine cell penetration sequence. SEQ ID NOS: 29 and 30 are control peptides.

FIG. 6 shows reduced cell viability of the pre B leukemic cell line 697, with lower levels of Bcl-2 (697-neo) or higher levels of Bcl-2 (697-Bcl2, engineered to express high levels of Bcl-2). The 2335 peptide, a Bad S/A peptide disulfide linked to penetratin, reduced cell viability at increasing concentrations of peptide. The control peptide 2336, the Bad 2S/A peptide with d-amino acids linked to penetratin, does not bind to Bcl-2 and was significantly less active than the 2335 peptide. In the 697-Bcl2 cells overexpressing Bcl-2, higher concentrations of the 2335 peptide were required to kill cells.

FIG. 7 shows the dose response curves plotted together for the 2335 peptide from FIG. 6 in low (697-neo) versus high (697-Bcl2) Bcl-2 expressing cells.

FIG. 8 shows reduced cell viability of acute myelogenous leukemia (AML) cells isolated from a patient (AML-728) treated with increasing concentrations of the 2335 peptide, in contrast to the control 2336 peptide.

FIG. 9 shows reduced cell viability of AML cells from a patient (AML-349) using the 2335 peptide relative to the control 2336 peptide.

### DETAILED DESCRIPTION OF THE INVENTION

The present invention provides a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1). In one embodiment, a modified BAD peptide or peptidomimetic includes alanine at positions corresponding to residues 16 and 22 of SEQ ID NO: 1. In another embodiment, a modified BAD peptide or peptidomimetic includes the amino acid sequence NLWAAQRYGRELRRMADEF VDAFKK (SEQ ID NO: 2) or a conservative variant or peptidomimetic thereof. In a further embodiment, a modified BAD peptide or peptidomimetic of the invention has an amino acid sequence consisting of SEQ ID NO: 2 or a peptidomimetic thereof.

Proteins of the Bcl-2 family are major regulators and effectors of programmed cell death, which culminates in the release of cytochrome c from mitochondria to activate Apaf-1 and activation of effector caspases. Members of the Bcl-2 family include both pro- and anti-apoptotic proteins and share homology in up to four conserved regions designated BCL-2 homology domains 1 through 4 (BH1-BH4; Adams and Cory, supra, 1998). The BCL-2 family can be divided into three main subclasses. The first is the anti-apoptotic proteins, which include BCL-2 and BCL-X_{L}, which are multidomain proteins sharing homology through BH 1 to BH4. The two subclasses of pro-apoptotic proteins are divided into 1) multidomain proteins such as BAX and BAK, which share homology in BH 1-3; and 2) the more distantly related “BH3-only” proteins, which share sequence homology within the amphipathic α-helical BH3 region required for their apoptotic function (Chittenden et al., EMBO J. 14:5589-5596 (1995); O'Connor et al., EMBO J. 17:384-395 (1998); Wang et al., Genes Dev. 10:2859-2869 (1996); and Zha et al., J. Biol. Chem. 27224101-24104 (1997)).

Multidomain pro-apoptotic proteins such as BAX and BAK release cytochrome c from mitochrondia and kill cells when triggered by the cell death signals. In viable cells, BAX and BAK exist as monomers. However, in response to a variety of cell death stimuli, inactive BAX, which is located in the cytosol or loosely attached to membranes, inserts as a homo-oligomerized multimer into the outer mitochondrial membrane (Eskes et al., 2000; Gross et al., 1998; and Wolter et al., 1997). Similarly, inactive BAK resides at mitochondria, where it undergoes an allosteric conformational change, including homooligomerization, in response to cell death signals (Griffiths et al., 1999; Wei et al., Genes Dev. 2060-2071 (2000)). Cells deficient in both BAX and BAK are resistant to a wide range of stimuli that normally result in cell death (Wei et al., Science 292:727-730 (2001)).

The BH3-only molecules include BAD as well as BID, NOXA, PUMA, BIK and BIM (Kelekar and Thompson, Trends Cell Biol. 8:324-330 (1998))). These proteins share sequence homology only in their amphipathic α-helical domain which is required for proapoptotic activity. In addition, the BH3 domain functions in binding to multidomain BCL-2 family members. Multiple binding assays including yeast two-hybrid, coimmunoprecipitation from detergent-solubilized cell lysates, and in vitro pull-down experiments, indicate that individual BH3 only molecules display some selectivity for multidomain BCL-2 binding partners (Boyd et al., Oncogene 11:1921-1928 (1995); O'Connor et al., EMBO J. 17:384-395 (1998); Oda et al., Science 288:1053-1058 (2000); Wang et al., supra, 1996; Yang et al., 1995). The BID protein binds proapoptotic BAX and BAK as well an anti-apoptotic BCL-2 and BCL-X_{L} (Wang et al., supra, 1996; Wei et al., supra, 2000). In contrast, BAD, as well as NOXA and BIM preferentially bind to anti-apoptotic BCL-2 family members (Boyd et al., supra, 1995; O'Connor et al., supra, 1998; Oda et al., supra, 2000; Yang et al., supra, 1995). These differences nowithstanding, expression of BID, BAD, BIM or NOXA results in activation of BAX and BAK. Moreover, the expression of these proteins in Bax/Bak doubly deficient cells demonstrates that BAX and BAK are absolutely required for induction of cell death (Cheng et al., Mol. Cell 8:705-711 (2001)); Zong et al., Genes Dev. 15:1481-1486 (2001)). Comparison of wild-type and mutant BCL-2 and BCL-X_{L} has shown that anti-apoptotic Bcl-2 family members sequester all of these BH3-only molecules in stable mitochondrial complexes, preventing the activation of BAX and BAK (Cheng et al., supra, 2001).

Analysis of synthetic BH3 peptides from BH3-only proteins has shown that the BID-like and BAD-like subsets appear to possess distinct functions. A BID-like subset “activates” pro-apoptotic BAK and BAX. In contrast, a BAD-like subset occupies the pocket of anti-apoptotic protein, BCL-2, thus sensitizing it to the availability of activating BH3 domains. Wild type BAD BH3 peptide (SEQ ID NO: 1) binds BCL-2 with a K_{d} of about 40 nM but does not have apoptotic activity (Letai et al., Cancer Cell 2:183-192 (2002)).

The modified BAD peptides and peptidomimetics of the invention have improved pharmacological properties as compared to the wild type BAD peptide SEQ ID NO: 1. In particular, the modified BAD peptides and peptidomimetics of the invention are effective at inducing cell death in cancer cells whereas wild type BAD peptide is not. As used herein, the term “modified BAD peptide or peptidomimetic” means a peptide or peptidomimetic which is structurally related to the wild type BAD peptide by having at least 60% amino acid identity with SEQ ID NO: 1 and which has enhanced affinity for Bcl-2 as compared to the wild type BAD peptide SEQ ID NO: 1.

TABLE 1
Wild type and modified Bad peptides

| | | |
|---|---|---|
| Peptide | SEQ ID NO: | Amino acid sequence |

| | | |
|---|---|---|
| Wild type | SEQ ID NO: 1 | NLWAAQRYGRELRRMSDEFVDSFKK |
| Bad | | |
| Bad 2Ser/ | SEQ ID NO: 2 | NLWAAQRYGRELRRMADEFVDAFKK |
| Ala | | |
| Bad 3A | SEQ ID NO: 3 | NLWAAQRYGREARRMADEFVDAFKK |
| control | | |
| Bad 4A | SEQ ID NO: 4 | NLWAAQRYGREARRMAAEFVDAFKK |
| control | | |

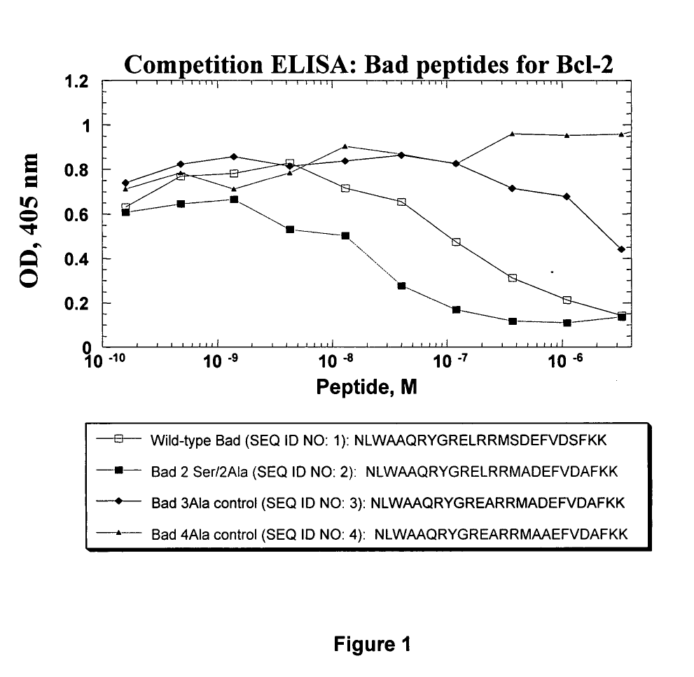

A modified BAD peptide or peptidomimetic has enhanced affinity for Bcl-2 as compared to the wild type BAD BH3 peptide (SEQ ID NO: 1). As disclosed herein in FIG. 1, the results of a competition ELISA indicate that modified BAD peptide SEQ ID NO: 2 has significantly higher binding affinity for Bcl-2 than the wild type BAD peptide SEQ ID NO: 1. Additional modified BAD peptides and peptidomimetics of the invention further include, without limitation, NLWAAQRYGRELRRMADEFVDSFKK (SEQ ID NO: 7) and NLWAAQRYGRELRRMSDEFVDAFKK (SEQ ID NO: 8).

Any of the above modified BAD peptides or peptidomimetics such as SEQ ID NO: 2, SEQ ID NO: 7 or SEQ ID NO: 8 can optionally include an alpha-helix nucleation site or other sequence which improves α-helical content. As a non-limiting example, a modified BAD peptide or peptidomimetic of the invention can be stabilized as an α-helix with a hydrazone link, which can act as a covalent mimic of a main-chain hydrogen bond. Such a hydrazone link (N—N═CH—CH₂CH₂) can be designed to replace the hydrogen bond (NH→O═C(R)NH) that forms between the main-chain amide proton of an upstream amino acid and the main-chain carbonyl oxygen of a downstream amino acid. The hydrogen bond is replaced by the N═CH double bond, and the associated peptide link replaced with an ethylene group. Given that an α-helix has a pattern of sequential hydrogen bonds between the amide of an upstream amino acid and the main-chain carbonyl oxygen of a residue 4 positions downstream, replacement of an amino-terminal (i+4→i) hydrogen bond with a hydrazone link favors an α-helical conformation.

A hydrazone link can be formed by the reaction of an activated acetal (J) with a hydrazine derivative (Z); J and Z are linking residues used to form a hydrazone link which can be represented in brackets as [JX₁X₂Z], with Z restricted to an α-methylene carbon to accommodate an α-helical conformation. Such amino-terminal hydrazone linkages useful in the peptides and peptidomimetics of the invention include, without limitation, [JLAZ], [JAAZ] and [JLPZ], as described, for example, in Cabezas and Satterthwait, J. Am. Chem. Soc. 121:3862-3875 (1999), and Cabezas et al., Biochem. 39:14377-14391 (2000). As non-limiting examples, a modified BAD peptide or peptidomimetic useful in the invention can be [JALZ] NLWAAQRYGRELRRMADEFVDAFKKC, [JAAZ]NLWAAQRYGRE LRRMADEFVDAFKKC, [JALZ]RYGRELRRMADEFVDAFKKC, or [JAAZ]RYGRELRRMADEFVDAFKKC. One skilled in the art understands that these and other modified BAD peptides stabilized in an α-helical conformation can be useful in treating leukemia or another cancer according to a method of the invention.

Any of a variety of assays can be useful for comparing the binding affinity of a modified BAD peptide or peptidomimetic of the invention to Bcl-2 with the binding affinity of the wild type BAD peptide SEQ ID NO: 1. Competition assays, for example using a BID BH3 peptide, can be useful for comparing the binding affinity of a modified BAD peptide or peptidomimetic of the invention to Bcl-2 with the binding affinity of SEQ ID NO: 1. As a non-limiting example, a competition ELISA assay can be performed, for example, as described in Cabezas et al., supra, 2000, with modifications as set forth in Example I. As a further non-limiting example, binding affinity to Bcl-2 can be determined essentially as set forth in Letai et al., supra, 2002. Briefly, the K_{d} for peptide or peptidomimetic binding to Bcl-2 can be determined using a GST-BCL-2 fusion protein lacking the carboxy-terminal transmembrane domain. GST-BCL-2 fusion protein can be expressed in bacteria by standard methods and purified on glutathione agarose beads. Peptides or peptidomimetics to be assayed for binding affinity can be synthesized with a fluorescein amino terminus using an AHA linker. Peptides or peptidomimetics at a concentration of 25 nM can be mixed with titrations of GST-BCL-2 in binding buffer (140 mM NaCl, 10 mM Tris, pH 7.4) at 37° C. An increase in fluorescence polarization measured on a Perkin-Elmer LS 50B luminescence spectrophotometer can be quantitated to calculate binding. A nonlinear fit to a sigmoidal dose-response curve utilizing the program Origin 6.0 can be used to determine K_{d} For quantitative BIDBH3 displacement assays, 25 mM fluoresceinated BIDBH3 can be mixed with 1 μM GST-BCL-2 in binding buffer. Increasing amounts of unlabeled BH3 peptides or peptidomimetics can be titrated in, with loss of fluorescence polarization indicating displacement of BIDBH3. Data can be fitted to a sigmoidal curve as described above, and the IC50 determined. One skilled in the art understands that this assay as well as any of a variety of other Bcl-2 binding assays can be useful including, without limitation, competition and non-competition binding assays, ELISA assays, fluorescence polarization assays and other assays known in the art.

The peptides and peptidomimetics of the invention are provided in isolated form. As used herein in reference to a peptide or peptidomimetic of the invention, the term “isolated” means a peptide or peptidomimetic that is in a form that is relatively free from material such as contaminating polypeptides, lipids, nucleic acids and other cellular material that normally is associated with the peptide or peptidomimetic in a cell or that is associated with the peptide or peptidomimetic in a library or crude preparation.

The invention provides, in part, a modified Bad peptide or peptidomimetic which is a conservative variant of SEQ ID NO: 2. As used herein, a “conservative variant” is an amino acid sequence in which a first amino acid is replaced by a second amino acid or amino acid analog having at least one similar biochemical property, which can be, for example, similar size, charge, hydrophobicity or hydrogen-bonding capacity. For example, a first hydrophobic amino acid can be conservatively substituted with a second (non-identical) hydrophobic amino acid such as alanine, valine, leucine, or isoleucine, or an analog thereof. Similarly, a first basic amino acid can be conservatively substituted with a second basic amino acid such as arginine or lysine, or an analog thereof. In the same way, a first acidic amino acid can be conservatively substituted with a second acidic amino acid such as aspartic acid or glutamic acid, or an analog thereof, or an aromatic amino acid such as phenylalanine can be conservatively substituted with a second aromatic amino acid or amino acid analog, for example, tyrosine.

As described above, the invention provides a variety of modified BAD peptides and peptidomimetics, which can be useful for inducing cell death in chronic lymphocytic leukemia and other cancer cells. As used herein, the term “peptide” is used broadly to mean peptides, proteins, fragments of proteins and the like. The term “peptidomimetic,” as used herein, means a peptide-like molecule that has the activity of the modified BAD peptide upon which it is structurally based. Such peptidomimetics include chemically modified peptides, peptide-like molecules containing non-naturally occurring amino acids, and peptoids, and have the Bcl-2 binding activity of the peptide upon which the peptidomimetic is structurally based (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861). The modified BAD peptides and peptidomimetics of the invention can be prepared by routine solid phase synthesis methods known in the art as described, for example, in Gordon and Balasubramanian, J. Chem. Technol. Biotechnol. 74:835-851 (1999).

A variety of peptidomimetics are known in the art including, without limitation, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; α,α-dialkylglycine or α-aminocycloalkane carboxylic acid; an N^{α}—C^{α} cyclized amino acid; an N^{α}-methylated amino acid; a β- or γ-amino cycloalkane carboxylic acid; an α,β-unsaturated amino acid; a β,β-dimethyl or β-methyl amino acid; a β-substituted-2,3-methano amino acid; an N—C^{δ} or C^{α}—C^{δ} cyclized amino acid; a substituted proline or another amino acid mimetic. A peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

The present invention further provides a composition containing a delivery agent and a modified BAD peptide or peptidomimetic which includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1). In one embodiment, a composition of the invention includes a modified BAD peptide or peptidomimetic which has at least 60% amino acid identity with SEQ ID NO: 1 and further includes alanine at positions corresponding to residues 16 and 22 of SEQ ID NO: 1. In another embodiment, a composition of the invention includes a modified BAD peptide or peptidomimetic which includes the amino acid sequence NLWAAQRYGRELRRMADEFVDAFKK (SEQ ID NO: 2) or a conservative variant or peptidomimetic thereof. In a further embodiment, a composition of the invention includes a modified BAD peptide or peptidomimetic which consists of the amino acid sequence SEQ ID NO: 2 or a peptidomimetic thereof. In yet another embodiment, the invention includes a modified BAD peptide or peptidomimetic that further comprises a penetratin (SEQ ID NO:27) fusion. An example of such a peptide is a penetratin fusion to the amino acid sequence of SEQ ID NO:2. It is understood that other BAD peptides or peptidomimetics disclosed herein can similarly be fused to a penetratin sequence. In a particular embodiment, the modified BAD peptide or peptidomimetic comprises the amino acid sequence NLWAAQRYGRELRRMADEFVDAFKKC-CRQIKIWFQNRRMKWKK (SEQ ID NO:31). Such a peptide can be amidated on the N-terminus and/or acetylated on the C-terminus (see Example III).

In a composition of the invention, the delivery agent can be covalently linked to, or non-covalently associated with, the modified BAD peptide or peptidomimetic. In one embodiment, a composition of the invention is a chimeric protein, peptide or peptidomimetic in which the delivery agent is operatively fused to the modified BAD peptide or peptidomimetic. Delivery agents useful in the compositions of the invention encompass, without limitation, polycationic homopolymers including polyarginine sequences such as _{D}Arg-_{D}Arg-_{D}Arg-_{D}Arg-_{D}Arg-_{D}Arg-_{D}Arg-_{D}Arg (SEQ ID NO: 5). In one embodiment, a composition of the invention includes the amino acid sequence NLWAAQRYGRELRRMADEFVDAFKKC-Ahx-_{D}Arg-_{D}Arg-_{D}Arg-_{D}Arg-_{D}Arg-_{D}Arg-_{D}Arg-_{D}Arg (SEQ ID NO: 6), where Ahx is aminohexanoic acid. In additional embodiments, a composition of the invention includes the amino acid sequence [JALZ] NLWAAQRYGRELRRMADEFVDAFKKC-Ahx-_{D}Arg-_{D}Arg-_{D}Arg-_{D}Arg-_{D}Arg-_{D}Arg-_{D}Arg-_{D}Arg, where Ahx is aminohexanoic acid, or [JAAZ]RYGRELRRMADEFVDAFKKC-Ahx-_{D}Arg-_{D}Arg-_{D}Arg-_{D}Arg-_{D}Arg-_{D}Arg-_{D}Arg-_{D}Arg, where Ahx is aminohexanoic acid.

Further provided herein are methods of treating leukemia in a patient by administering to the patient a composition which contains a delivery agent and a modified BAD peptide or peptidomimetic that includes an amino acid sequence having at least 60% amino acid identity with SEQ ID NO: 1, where the modified BAD peptide or peptidomimetic has enhanced affinity for Bcl-2 as compared to wild type BAD peptide (SEQ ID NO: 1). Leukemias to be treated according to a method of the invention include, without limitation, chronic lymphocytic leukemia. Any of a variety of modified BAD peptide or peptidomimetics can be useful in the methods of the invention including, without limitation, a modified BAD peptide or peptidomimetic having at least 60% amino acid identity with SEQ ID NO: 1 and further including alanine at positions corresponding to residues 16 and 22 of SEQ ID NO: 1. In one embodiment, a method of the invention is practiced with a composition containing a modified BAD peptide or peptidomimetic which includes the amino acid sequence NLWAAQRYGRELRRMADEFVDAFKK (SEQ ID NO: 2) or a peptidomimetic thereof. In another embodiment, a method of the invention is practiced with a composition containing a modified BAD peptide or peptidomimetic having an amino acid sequence which consists of NLWAAQRYGRELRRMADEFV DAFKK (SEQ ID NO: 2) or a peptidomimetic thereof. In yet another embodiment, a method of the invention is practiced with a modified BAD peptide or peptidomimetic that further comprises a penetratin (SEQ ID NO:27) fusion. An example of such a peptide is a penetratin fusion to the amino acid sequence of SEQ ID NO:2. It is understood that other BAD peptides or peptidomimetics disclosed herein can similarly be fused to a penetratin sequence and used in methods of the invention. In a particular embodiment, the modified BAD peptide or peptidomimetic comprises the amino acid sequence NLWAAQRYGRELRRMADEFVDAFKKC-CRQIKIWFQNRRMKWKK (SEQ ID NO:31). Such a peptide can be amidated on the N-terminus and/or acetylated on the C-terminus (see Example III).

In the methods of the invention, the delivery agent can be covalently linked to the modified BAD peptide or peptidomimetic, or non-covalently associated with the modified BAD peptide or peptidomimetic. Furthermore, the composition can be, for example, a chimeric protein, peptide or peptidomimetic in which the delivery agent is operatively fused to the modified BAD peptide or peptidomimetic. Delivery agents useful in the methods of the invention include, without limitation, polycationic homopolymers such as polyarginine sequences, for example, _{D}Arg-_{D}Arg-_{D}Arg-_{D}Arg-_{D}Arg-_{D}Arg-_{D}Arg-_{D}Arg (SEQ ID NO: 5). In one embodiment, a method of the invention is practiced by administering a composition which includes the amino acid sequence NLWAAQRYGRELRRMADEFVDAFKKC-Ahx-_{D}Arg-_{D}Arg-_{D}Arg-_{D}Arg-_{D}Arg-_{D}Arg-_{D}Arg-_{D}Arg (SEQ ID NO: 6), where Ahx is aminohexanoic acid.

In the compositions and methods of the invention, a delivery agent facilitates uptake of a modified BAD peptide or peptidomimetic into a cell such as a leukemia cell. The delivery agent can be covalently linked to the modified BAD peptide or peptidomimetic or can be non-covalently associated with the modified BAD peptide or peptidomimetic. As non-limiting examples, delivery agents useful in the compositions of the invention include polycationic homopolymers such as polyarginine, antennapedia proteins, HIV TAT proteins, herpes simplex virus VP22 proteins, and active fragments thereof, as well as delivery agents such as Chariot™ and other MPG peptides, each of which is discussed further below.

As used herein, the term “delivery agent” means any molecule that enables or enhances internalization of an associated or linked modified BAD peptide or peptidomimetic into a cell. Delivery agents are known in the art and include, but are not limited to, cell-permeant peptides and peptidomimetics and protein transduction peptides and peptidomimetics. The term delivery agent encompasses, without limitation, proteins, peptides, peptidomimetics, small molecules, nucleic acid molecules, liposomes, lipids, viruses and retroviruses. It is understood that the term “delivery agent” encompasses molecules that are internalized by any mechanism, including delivery agents which function via receptor-mediated endocytosis and those which are independent of receptor-mediated endocytosis. It further is understood that a composition of the invention generally is not retained in intracellular vesicles upon internalization but, rather, is eventually delivered, for example, to the cytoplasm. Thus, the term delivery agent encompasses, without limitation, molecules that transport associated or linked substrate to the cell cytoplasm.

A variety of delivery agents can be covalently linked to a modified BAD peptide or peptidomimetic in a composition of the invention, including, without limitation, cell permeant peptides, phosphopeptides, peptides containing D-amino acids, protein transduction peptides, and other denatured or folded, modified or unmodified, and naturally occurring or synthetic proteins, peptides and peptidomimetics. Such delivery agents include, without limitation, nuclear and secreted proteins and active fragments and analogs thereof. In particular embodiments, a delivery agent useful in the invention is a peptide or peptidomimetic having a length of less than 50 residues, a length of less than 40 residues, a length of less than 30 residues, a length of less than 20 residues, or a length of less than 10 residues. In other embodiments, a delivery agent useful in the invention is a predominantly basic peptide or peptidomimetic, a polycationic homopolymeric peptide or peptidomimetic, or a peptide or peptidomimetic containing D-amino acids such as a poly-D-arginine sequence. In still other embodiments, a delivery agent useful in the invention is a predominantly hydrophobic peptide or peptidomimetic; an α-helical peptide or peptidomimetic such as an amphipathic α-helical peptide or peptidomimetic; or an amphipathic peptide or peptidomimetic such as a basic amphipathic peptide or peptidomimetic. In a further embodiment, a delivery agent useful in the invention is a denatured peptide or peptidomimetic, which is linked to a denatured or folded modified BAD peptide or peptidomimetic.

A delivery agent useful in the invention can be a synthetic sequence that shares one or more characteristics of a naturally occurring delivery agent such as a protein transduction domain (PTD). Such delivery agents include, but are not limited to, polycationic homopolymers such as L- and D-arginine oligomers (Mitchell et al., J. Peptide Res. 56:318-325 (2000)). For example, polymers of L- or D-arginine containing 6 or more amino acids and related peptoids can be useful as delivery agents in the methods of the invention (Mitchell et al., J. Peptide Res. 56:318-325 (2000); Wender et al., Proc. Natl. Acad. Sci., USA 97:13003-13008 (2000)). As one non-limiting example, a 7-mer of D-arginine can be useful as a delivery agent in a composition or method of the invention (see Example II).

Delivery agents suitable for use in the compositions of the invention include, without limitation, ciliary neurotrophic factor (CNTF) or an active fragment thereof; caveolin or an active fragment thereof; interleukin-1β (IL-1β) or an active fragment thereof; thioredoxin or an active fragment thereof; Antennapedia or an active fragment thereof such as penetratin-1 (SEQ ID NO: 27); fibroblast growth factor-1 (FGF-1) or an active fragment thereof; Engrailed or an active fragment thereof; Hoxa-5 or an active fragment thereof; Kaposi fibroblast growth factor (kFGF) or an active fragment thereof, for example, AAVALLPAVLLALLAP (SEQ ID NO: 9); human β3 integrin or the hydrophobic signal sequence or other active fragment thereof; a nuclear localization sequence such as TPPKKKRKVEDP (SEQ ID NO: 10); FGF-2 or an active fragment thereof; transportan or an active fragment thereof such as GWTLNSAGYLLGKINLKALAALAKKIL (SEQ ID NO: 11); lactoferrin or an active fragment thereof; VP22 or an active fragment thereof; HIV type I transactivator (HIV TAT) or an active fragment thereof such as YGRKKRRQRRR (SEQ ID NO: 12); or a heat shock protein such as HSP70 or an active fragment thereof. These and additional delivery agents are well known in the art as described, for example, in Ho, Cancer Res. 61:474-477 (2001); Schwarze and Dowdy, Trends Pharmacol. Sci. 21:45-48 (2000); Prochiantz, Curr. Opin. Cell Biol. 12:400-406 (2000); Ford et al., Gene Therapy 8:1-4 (2001); Dunican and Doherty, Biopolymers Peptide Sci. 60:45-60 (2001); Schwartz and Zhang, Curr. Opin. Mol. Ther. 2:162-167 (2000); and Wang et al., Cancer Res. 60:1498-1502 (2000).

As a non-limiting example, the invention can be practiced with a delivery agent which is a homeoprotein or an active fragment thereof, for example, a homeodomain or an active fragment thereof. Homeoproteins are helix-turn-helix proteins that contain a DNA-binding domain of about 60 residues, denoted the homeodomain. A variety of homeoproteins, homeodomains and active fragments thereof can be delivery agents useful in the invention including, without limitation, Antennapedia, Engrailed1 (En1), Engrailed2 (En2), Hoxa-5, Hoxc-8, Hoxb-4 and Knotted-1 (KN1). As an example, En1 and En1 have been expressed in COS7 cells, where they are first secreted and then internalized by other cells. See, for example, Prochiantz, supra, 2000.

A delivery agent can be the homeodomain protein, Antennapedia, or an active fragment thereof. Antennapedia is a member of a family of developmentally important Drosophila homeoproteins which translocate across neuronal membranes. The third helix of the Antennapedia homeodomain, the 16 residue peptide “penetratin-1” (SEQ ID NO: 27), is internalized into live cells. The internalization occurs both at 37° C. and 4° C., indicating that delivery is neither receptor-mediated nor energy-dependent. Additional delivery agents include peptides and peptidomimetics related in sequence to Penetratin-1 such as, without limitation, one of the peptides shown below in Table 2 or another penetratin-derived peptide or peptidomimetic, including a retroinverse or all D-acid peptide or peptidomimetic, or a related but non-α-helical peptide or peptidomimetic (see, for example, Prochiantz, supra, 2000). In one embodiment, such a penetratin-derived peptide retains the tryptophan, phenylalanine and glutamine residues of penetratin-1 (SEQ ID NO: 27).

TABLE 2

Penetratin-derived peptides useful
as delivery agents

| | | |
|---|---|---|
| Peptide | SEQ ID NO: | Amino acid sequence |

| | | |
|---|---|---|
| Penetratin-1 | SEQ ID NO: 27 | RQIKIWFQNRRMKWKK |
| 58-43 | SEQ ID NO: 13 | KKWKMRRNQFWIKIQR |
| Pro50 | SEQ ID NO: 14 | RQIKIWFPNRRMKWKK |
| 3Pro | SEQ ID NO: 15 | RQPKIWFPNRRMPWKK |
| Met-Arg | SEQ ID NO: 16 | RQIKIWFQNMRRKWKK |
| 7Arg | SEQ ID NO: 17 | RQIRIWFQNRRMRWRR |
| W/R | SEQ ID NO: 18 | RRWRRWWRRWWRRWRR |

A delivery agent useful in the invention also can be a HIV trans-activator (TAT) protein or an active fragment thereof. Such a delivery agent can include, for example, a sequence identical or similar to residues 47-57 or 47-59 of HIV TAT (Schwartz et al., Science 285:1569-1572 (1999); and Ho et al., supra, 2001). As an example, fusion proteins including residues 47-57 of HIV TAT (YGRKKRRQRRR; SEQ ID NO: 12) cross the plasma membrane of human and murine cells in vitro and in vivo (Schwartz and Zhang, supra, 2000); and a variety of proteins from 15 to 120 KDa have been shown to retain biological activity when fused to a HIV TAT delivery agent. An HIV TAT delivery agent can be positively charged and can function, for example, in an energy-, receptor-, transporter- and endocytosis-independent manner to deliver a covalently linked modified BAD peptide or peptidomimetic to, for example, 90-100% of target cancer cells.

A delivery agent useful in the invention also can be a herpes simplex virus VP22 protein or an active fragment thereof. In one embodiment, the delivery agent is an HSV type 1 (HSV-1) VP22 protein or active fragment thereof. HSV VP22, a nuclear transcription factor, can cross the plasma membrane through non-classical endocytosis and can enter cells independent of GAP junctions and physical contacts. As a fusion with a variety of different proteins, HSV VP22 results in uptake into cells of different types including terminally differentiated cells (Ford et al., supra, 2001; Schwartz and Zhang, supra, 2000) and has been shown to deliver a linked peptide to 90-100% of cultured cells.

Delivery agents useful in the invention further include those which correspond to, or are derived from, a hydrophobic signal sequence. Such a delivery agent can be, for example, the Kaposi fibroblast growth factor (kFGF) or an active fragment thereof such as AAVALLPAVLLALLAP (SEQ ID NO: 9); human β3 integrin or an active fragment thereof; or another hydrophobic delivery agent such as one of those described in Dunican and Doherty, supra, 2001. One skilled in the art understands that additional delivery agents also are useful in the compositions and methods of the invention. Such delivery agents include, without limitation, basic peptides and peptidomimetics; basic α-helical peptides and peptidomimetics; peptides and peptidomimetics with optimized arginine alignment or optimized α-helical character as compared to a naturally occurring protein transduction domain such as residues 47-57 of HIV TAT (Ho et al., supra, 2001; WO 99/29721); SCWKₙ (SEQ ID NO: 19); (LARL)ₙ(SEQ ID NO: 20); HA2; RGD; K₁₆RGD (SEQ ID NO: 21); loligomer; AlkCWK₁₈ (SEQ ID NO: 22); DiCWK₁₈ (SEQ ID NO: 23); DipaLytic; Plae (SEQ ID NO: 24); Kplae (SEQ ID NO: 25) and other delivery agents known in the art or which can be prepared by routine methods. The skilled person understands that these and other naturally occurring and synthetic delivery agents can be useful for facilitating cellular uptake of a modified BAD peptide or peptidomimetic in the methods of the invention.

A delivery agent useful in the invention also can be an agent that enables or enhances cellular uptake of a non-covalently associated modified BAD peptide or peptidomimetic. Such delivery agents include, without limitation, peptides containing independent hydrophobic and hydrophilic domains; MPG peptides such as GALFLGFLGAAGSTMGAWSQPKSKRKV (SEQ ID NO: 26) and other MPG peptides, which are derived from both the nuclear localization sequence (NLS) of SV40 large T antigen and the fusion peptide domain of HIV-1 gp4; and amphipathic peptides such as Pep-1. These and related delivery agents that function in the absence of covalent linkage, sometimes known as “protein transfection products,” are well known in the art as described, for example, in Morris et al., Nucl. Acids Res. 27:3510-3517 (1999); Morris et al., J. Biol. Chem. 274:24941-24946 (1999); and Morris et al., Nature Biotech.19:1173-1176 (2001). Such peptide delivery agents can be prepared by routine methods and are commercially available. As an example, the Chariot™ product is commercially available from Active Motif (Carlsbad, Calif.).

Previous work has suggested that agents which reduce Bcl-2 expression can decrease the survival of cancer cells, for example, when used in combination with standard anti-cancer therapy. Antisense Bcl-2 oligonucleotides have shown benefit in a SCID mouse model of B cell lymphoma (Cotter et al., Oncogene 9:3049-3055 (1994)); in a mouse model of mammary carcinoma (Miyake et al., Cancer Res. 59:4030-4034 (1999)); and in the LNCaP prostate cancer model (Gleave et al., Clin. Cancer Res. 5:2891-2898 (1999)). Chemosensitization by antisense Bcl-2 has been demonstrated in several models including a SCID model of human melanoma treated with dacarbazine (Jansen et al., Nat. Med. 4:232-234 (1998)); LNCaP prostate cancer treated with paclitaxel (Leung et al., Int. J. Cancer 91:846-850 (2001)); MDA435/LCC6 breast cancer xenografts treated with doxorubicin (Lopes de Menezes et al., Clin. Cancer Res. 6:2891-2902 (2000)); human gastric cancer treated with cisplatin (Wacheck et al., J. Mol. Med. 79:587-593 (2001)); and Merkel cell carcinomas (Schlagbauer-Wadl et al., J. Invest. Dermatol. 114:725-730 (2000)). In addition, Bcl-2 antisense oligodeoxynucleotides have shown promise when combined with standard anti-cancer agents in Phase I-II trials of metastatic melanoma; small cell lung cancer; prostate cancer; and acute leukemia (Jansen et al., Lancet 356:1728-1733 (2000); Rudin et al., Ann. Oncol. 13:539-545 (2002); Chi et al., Clin. Cancer Res. 7:3920-3927 (2001); Tolcher, Semin. Oncol. 28:67-70 (2001); and Marcucci et al., Blood 101:425-432 (2003)), with Phase III studies in progress in chronic lymphocytic leukemia, multiple myeloma and non-small cell lung cancer. These early results indicate that agents which diminish Bcl-2 expression or counteract Bcl-2 activity, such as the peptides, peptidomimetics and compositions of the invention, can enhance apoptosis and be useful for treating cancer.

The methods of the invention can be useful for treating any of a variety of types of cancer including cancers including, without limitation, primary and metastatic cancer and drug-resistant cancer. As non-limiting examples, the methods of the invention can be useful for treating cancers in which Bcl-2 expression is upregulated, such as those set forth in Table 3. Cancers which can be treated according to a method of the invention include, yet are not limited to, leukemias such as acute myelogenous leukemia, acute lymphocytic leukemia, chronic myelogenous leukemia and chronic lymphocytic leukemia and acute leukemia; lymphomas including Hodgkin's lymphoma and non-Hodgkin's lymphoma; myelomas such as multiple myeloma; lung cancers including non-small cell lung cancer and small cell lung cancer; melanomas such as metastatic melanoma; prostate cancers; colorectal cancer; breast cancers; pancreatic cancers; urogenital cancers; ovarian cancers; brain cancers including medulloblastomas, gliomas and oligodendrogliomas; and esophageal cancers (Manion and Hockenberry, Cancer Biol. & Therapy 2 (Suppl. 1): S105-S114 (2003)). One skilled in the art understands that these and other cancers, including but not limited to those in which Bcl-2 expression is upregulated, can be treated with a modified Bad peptide or peptidomimetic of the invention.

TABLE 3
Percentages of Common Human Cancers with Elevated Levels of Bcl-2

| Tumor | Bcl-2 | | Reference |
|---|---|---|---|
| Lung | NSCLC | squamous 25% | Pezzella et al., N. Engl. J. Med. 329:690-4 |
| | | adenoca 12% | (1993). |
| | SCLC | 83-90% | Reeve et al., Br. J. Cancer. 73:1193-200 |
| | | | (1996). |
| Colorectal | Adenoma | 65-98% | Yang et al., Anticancer Res. 19:727-30 |
| | Carcinoma | 46-60% | (1999); Hao et al., Pathobiology 65:140-5 |
| | | | (1997). |
| Breast | 80% | | Krajewski et al., Endocr. Relat. Cancer. 6:29- |
| | | | 40(1999) |
| Pancreas | 23% | | Miyamoto et al., Oncology 56:73-82 (1999) |
| Urogenital | Bladder | 24% | Glick et al., J. Urol. 155:1754-7 (1996) |
| | Renal | 53% | Ghanem et al., Cancer 85:1557-63 (2001) |
| Lymphoma | Hodgkin's | 47-65% | Rassidakis et al., Blood 100:3935-41 (2002) |
| | NHL | 9-57% | Bairey et al., Clin. Cancer. Res. 5:2860-6 |
| | | | (1999); |
| | | | Gascoyne et al., Blood 90:244-51 (1999); |
| | Burkitt's | negative | Xerri et al., |
| | | | Leukemia 13:1548-53 (1999) |
| | | | Gala et al., Ann. Hematol. 69:17-24 (1994) |
| Leukemia | AML | 13-20% | Schaich et al., Haematologica 86:470-7 |
| | | | (2001); |
| | ALL | 89-92% | Kornblau et al., Clin. Cancer. Res. 5:1758-66 |
| | | | (1999) |
| | CML | 33-54% | Salomons et al., Leukemia 13:1574-80 (1999); |
| | CLL | 70-95% | Soslow, et al. Hum. Pathol. 28:1158-65 |
| | | | (1997) |
| | | | Ravandi et al., Cancer 91:1964-72 (2001) |
| | | | Hanada et al., Blood 82:1820-8 (1993) |
| Liver | Rare | | Charlotte et al., Am. J. Pathol. 144:460-5 |
| | | | (1994) |
| Ovary | 30-39% | | Schaich et al., supra 2001; |
| | | | Kornblau et al., Clin. Cancer. Res. 5:1758-66 |
| | | | (1999) |
| Brain | Medulloblastoma | 5-25% | Sarkar et al., 59:49-61; Krajewski et al., Am. |
| | | | J. Pathol. 150:805-14 (1997) |
| | Gliomas | 28-92% | Krajewski et al., Am. J. Pathol. 150:805-14 |
| | | | (1997); |
| | | | Ellison et al., Neuropathol. Appl. Neurobiol. |
| | Oligodendrogliomas | 16-60% | 21:352-61 (1995) |
| | | | Delgado et al., Neuropathol. Appl. Neurobiol. |
| | | | 25:400-7 (1999); Deininger et al., Cancer |
| | | | 86:1832-9 (1999) |
| Esophagus | SCC | 45% | Koide et al., Surg. Today 27:27:685-91 (1997) |
| | Adenocarcinoma | 20-40% | Katada et al. Arch. Surg. 132:728-33 (1997) |
| Myeloma | 43% | | Tu et al., Cancer Res. 58:256-62 (1998) |

In one embodiment, the methods of the invention are useful for treating a subject with leukemia. As used herein, the term “leukemia” means a neoplastic disease of leukocytes. Leukemias are characterized by distorted proliferation or development of leukocytes and include, without limitation, leukemias involving an increase in the number of leukocytes in the blood. Leukemias which can be treated according to a method of the invention include both acute and chronic leukemias, terms referring to the degree of cell differentiation, and further include leukemias of any type of leukocyte.

The methods of the invention can be useful for treating chronic leukemias, in which the involved cell line is well-differentiated. Forms of chronic leukemia to be treated according to a method of the invention include, without limitation, chronic granulocytic leukemia (chronic myeloid leukemia; chronic myelocytic leukemia; chronic myelogenous leukemia), which occurs mainly between the ages of 25 and 60 and is usually associated with chromosomal abnormality; chronic lymphocytic leukemia (CLL), which is mainly seen in the elderly and usually involves malignant differentiated B lymphocytes; chronic myelomonocytic leukemia, a slowly progressing form of chronic leukemia, typically seen in the elderly and which may progress to acute myelomonocytic leukemia; chronic eosinophilic leukemia, a form of chronic leukemia which may follow an acute course; and hairy cell leukemia (leukemic reticuloendotheliosis).

The methods of the invention also can be useful for treating acute leukemia, in which the involved cell line shows little or no differentiation and usually consists of blast cells. Acute leukemias to be treated according to a method of the invention include, without limitation, acute lymphocytic leukemia and acute myelogenous leukemia (acute granulocytic leukemia). As a non-limiting example, the methods of the invention can be useful for treating any of a variety of forms of acute lymphoblastic leukemia (acute lymphocytic leukemia; ALL), an acute leukemia which primary effects young children, including the rare B-cell type (Burkitt-like), common type, null cell type, pre-B-cell type and T-cell type. The methods of the invention also can be useful for treating any of a variety of forms of acute myelogenous leukemia (AML), also known as acute myelocytic leukemia and acute nonlymphocytic leukemia, which typically effects middle-aged to elderly individuals, including, without limitation, acute undifferentiated leukemia (AUL; acute blast cell leukemia), acute myeloblastic leukemia, acute promyelocytic leukemia, acute myelomonocytic leukemia, acute monocytic leukemia, acute erythroleukemia, and acute megakaryblastic leukemia, also denoted acute megakaryocytic leukemia. One skilled in the art understands that these as well as other forms of chronic and acute leukemia of varying severity can be treated according to a method of the invention.

One skilled in the art of oncology understands that a modified BAD peptide or peptidomimetic, or related composition of the invention, can be administered in conjunction with another anti-cancer agent. Anti-cancer agents include cytotoxic agents, which are molecules that result in cell death by any mechanism and encompass, without limitation, taxanes such as docetaxel (Taxotere; Aventis Pharmaceuticals, Inc.; Parsippany, N.J.) and paclitaxel (Taxol; Bristol-Myers Squibb; Princeton, N.J.; (Chan et al., J. Clin. Oncol. 17:2341-2354 (1999), and Paridaens et al., J. Clin. Oncol. 18:724 (2000)); anthracyclins such as doxorubicin, idarubicin or daunorubicin (Stewart and Ratain, In: “Cancer: Principles and practice of oncology” 5th ed., chap. 19 (eds. DeVita, Jr., et al.; J. P. Lippincott 1997); Harris et al., In “Cancer: Principles and practice of oncology,” supra, 1997)); alkylating agents such as melphalan or chlorambucil; vinca alkaloids such as vindesine, vinblastine or vinorelbine; anti-metabolites such as 5-fluorouracil, 5-fluorouridine or a derivative thereof; platinum agents such as cisplatin or carboplatin (Crown, Seminars in Oncol. 28:28-37 (2001)); DNA targeting agents including alkylating agents, agents that intercalate into DNA, and agents which result in double-stranded DNA breaks such as cyclophosphamide, melphalan, mitomycin C, bizelesin, cisplatin, doxorubicin, etoposide, mitoxantrone, SN-38, Et-743, actinomycin D, bleomycin, TLK286 and SGN-15 (Hurley, supra, 2002); steroids such as methotrexate; antibiotics such as adriamycin; antimicrobial peptides; mitomycin-C, adriamycin, ifosfamide and ansamycins; and other anti-cancer agents, which are molecules that inhibit the proliferation, growth, life-span or metastatic activity of cancer cells. Where an anti-cancer agent is administered in addition to a modified BAD peptide or peptidomimetic or composition of the invention, it is understood that the anti-cancer agent can be administered prior to, simultaneously with, or subsequent to, the modified BAD peptide or peptidomimetic or composition of the invention. Similarly, the anti-cancer agent can be administered by a same or different route of administration and in the same or different pharmaceutical composition as the modified BAD peptide or peptidomimetic or composition of the invention.

A modified BAD peptide or peptidomimetic or composition of the invention generally is administered in a pharmaceutical composition. A pharmaceutical composition useful in the invention includes the modified BAD peptide or peptidomimetic or composition and further can include, if desired, an excipient such as a pharmaceutically acceptable carrier or a diluent, which is any carrier or diluent that has substantially no long term or permanent detrimental effect when administered to a patient. Such an excipient generally is mixed with active peptide, peptidomimetic or composition, or permitted to dilute or enclose the active peptide, peptidomimetic or composition. A carrier can be a solid, semi-solid, or liquid agent that acts as an excipient or vehicle for the active peptide, peptidomimetic or composition. Examples of pharmaceutically acceptable carriers and diluents include, without limitation, water, such as distilled or deionized water; saline; and other aqueous media. It is understood that the active ingredients can be soluble or can be delivered as a suspension in the desired carrier or diluent.

A pharmaceutical composition further can include, if desired, one or more agents such as emulsifying agents, wetting agents, sweetening or flavoring agents, tonicity adjusters, preservatives, buffers or anti-oxidants. Tonicity adjustors useful in a pharmaceutical composition include salts such as sodium chloride, potassium chloride, mannitol or glycerin and other pharmaceutically acceptable tonicity adjustors. Preservatives useful in a pharmaceutical composition include, without limitation, a stabilized oxy chloro composition, for example, PURITE®, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in a pharmaceutical composition are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition containing a modified BAD peptide, peptidomimetic or composition of the invention.

A modified BAD peptide, peptidomimetic or composition of the invention is administered to a patient in an effective amount. Such an effective amount generally is the minimum dose necessary to achieve the desired therapeutic effect, which can be, for example, that amount roughly necessary to reduce one or more symptoms of the patient's cancer or to achieve one or more other clinical indicators of improvement. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity and type of cancer, the age and weight of the patient, the patient's general physical condition, and the route of administration. Where repeated administration is used, the frequency of administration depends, in part, on the half-life of the modified BAD peptide, peptidomimetic or composition. Extended release and slow-release formulations can be useful in the invention and include, for example, dermal patches, formulations for deposit on or under the skin and formulations for intramuscular injection. The patient being treated can be any animal having cancer, including mammals such as humans.

Various routes of administration can be useful for treating leukemia or another cancer according to a method of the invention. A pharmaceutical composition useful in the methods of the invention can be administered to a patient by any of a variety of means depending, for example, on the type and severity of cancer to be treated, and the history, risk factors and symptoms of the subject. Routes of administration suitable for use in the methods of the invention include both systemic and local administration. As non-examples, a modified BAD peptide, peptidomimetic or composition can be administered orally or by subcutaneous pump; by dermal patch; by intravenous, subcutaneous or intramuscular injection; by topical drops, creams, gels or ointments; as an implanted or injected extended release formulation; by subcutaneous minipump or other implanted device; by intrathecal pump or injection; or by epidural injection. It is understood that the frequency and duration of dosing will be dependent, in part, on the relief desired and the half-life of the modified BAD peptide or peptidomimetic.

In one embodiment, a method of the invention is practiced by peripheral administration of the modified BAD peptide, peptidomimetic or composition. Peripheral administration encompasses any route of administration other than direct administration to the spine or brain and can be local or systemic. Local administration results in significantly more of a pharmaceutical composition being delivered to the site of local administration than to regions distal to the site of administration. Systemic administration results in delivery of a pharmaceutical composition to essentially the entire peripheral nervous system of the subject and can also result in delivery to the central nervous system depending on the properties of the composition. Routes of peripheral administration useful in the methods of the invention encompass, without limitation, oral administration, intravenous or other injection, topical administration, and implanted minipumps or other extended release devices or formulations. A pharmaceutical composition containing a modified BAD peptide, peptidomimetic or composition can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. One skilled in the art understands that these and other pharmaceutical compositions and routes of administration known in the art of oncology can be useful in the methods of the invention.

The following examples are intended to illustrate but not limit the present invention.

### EXAMPLE I

### Induction of Apoptosis in Cancer Cells With Modified BAD Peptides

A. Binding Affinity of Modified BAD Peptides to Bcl-2

Several wild type, modified and control BAD peptides were prepared by standard solid phase synthesis: wild type BAD peptide NLWAAQRYGRELRRM SDEFVDSFKK (SEQ ID NO: 1); modified BAD peptide 2Ser/Ala NLWAAQRYGRELRRMADEFVDAFKK (SEQ ID NO: 2); BAD 3A control NLWAAQRYGREARRMADEFVDAFKK (SEQ ID NO:3) and BAD 4A control NLWAAQRYGREARRMAAEFVDAFKK (SEQ ID NO: 4). See Table 1 above.

To determine binding affinity to Bcl-2, competition ELISA assays were performed essentially as described in Cabezas et al., supra, 2001, with the following components. Briefly, acetyl-Cys(biotin-BMCC)-Ahx-Bad peptide (50 ng peptide/50 μl ELISA buffer) was adsorbed to 96-well neutraAvidin coated microtiter plates. Competition peptides were subsequently mixed with 50 ng GST-Bcl2(1-205; Santa Cruz Biotechnology; Santa Cruz, Calif.) in 50 μl ELISA buffer per titer well and incubated for one hour at 37° C. Wells were washed and treated with 50 μl of a 1/4000 dilution of anti-GST Mab 7E5 (from ascites fluid provided by Shinichi Kitada) in ELISA buffer for one hour at 37° C. Quantification of peptide-bound GST-Bcl2/Mab7E5 complex was carried out with alkaline-phosphatase-conjugated goat anti-rabbit IgG and p-nitrophenylphosphate substrate as described in Cabezas et al., supra, 2001.

As shown in FIG. 1, the results of the competition ELISA indicate that the modified BAD peptide SEQ ID NO: 2 has significantly higher binding affinity for Bcl-2 than the wild type BAD peptide SEQ ID NO: 1. These results indicate that serine to alanine or other alterations in the wild type primary BAD BH3 peptide sequence can produce modified peptides with enhanced binding affinity for Bcl-2.

B. Induction of Apoptosis in chronic Lymphocytic Leukemia (CLL) cells by Modified BAD Peptides

Peripheral blood mononuclear cells (PBMC) from patients with chronic lymphocytic leukemia (CLL) were obtained from the CLL Research Consortium (CRC) tissue bank. Briefly, heparinized peripheral blood was obtained from patients diagnosed with B-CLL according to standard criteria (Chenson et al., Blood 87:4990-4997 (1996)). Lymphocytes were isolated by Ficoll density-gradient centrifugation and verified by immunofluorescence flow cytometry to be composed of greater than 95% CD5/CD19/CD23 triple positive B cells. CLL samples were incubated at 1×10⁶ cells/ml in RPMI media supplemented with 10% FBS (fetal bovine serum) and 1 mM L-glutamine and antibiotics (penicillin/streptomycin) at 37° C. with 5% CO₂ for 24 hours. CLL cells were electroporated with a dimethyl sulfoxide (DMSO) control or peptides essentially as described in Eksioglu-Demiralp et al., J. Immunol. Methods 275:41-56 (2003), and Schimmer et al., Cancer Res. 63:1242-1248 (2003).

For evaluation of apoptosis, at least 5×10⁵ cells were recovered by centrifugation and double-stained with Annexin V-FITC, an early marker of apoptosis, and propidium iodide (PI), a nucleic acid-binding vital dye, followed by flow-cytometric analysis using the FL-1 and FL-3 channels of a Becton Dickinson flow cytometer (FACSort; San Jose, Calif.).

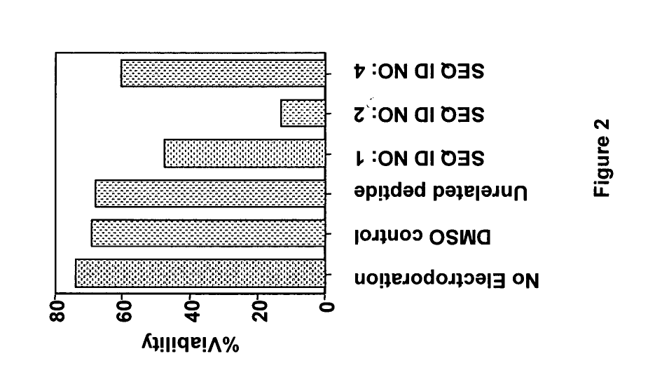

As shown in FIG. 2, the cell viability of chronic lymphocytic leukemia (CLL) cells was significantly reduced when cells were electroporated with the modified BAD 2Ser/Ala peptide SEQ ID NO: 2. This reduction in viability was much greater than the minimal or insignificant changes in viability produced by wild type BAD peptide SEQ ID NO: 1 or control BAD peptide SEQ ID NO: 4. These results demonstrate that modified BAD peptides and peptidomimetics such as the 2Ser/Ala peptide SEQ ID NO: 2 can reduce viability of cancer cells such as leukemic cancer cells.

Furthermore, as shown in Table 4, cell viability was dramatically reduced in all the patient CLL samples electroporated with the modified BAD peptide, Bad 2Ser/Ala (SEQ ID NO: 2), but was minimally effected in cells electroporated with the wild type BAD peptide SEQ ID NO: 1 or the control BAD peptide SEQ ID NO: 4. In sum, these results demonstrate that the enhanced Bcl-2 binding affinity observed with modified BAD peptides can induce apoptosis in cancer cells such as chronic lymphocytic leukemia cells.

TABLE 4
Induction of Apoptosis in CLL Cells with Bad 2Ser/2Ala Peptide
(SEQ ID NO: 2)

| | Patient Samples | | | | | | |
|---|---|---|---|---|---|---|---|
| | TJK | TJK | | | | | |
| | 340 | 340 | TJK | TJK | JCB | | MA |
| Peptides | #1 | #2 | 328 | 375 | 178 | Av. | G15 |
| None | 61 | 62 | 67 | 59 | 71 | 64 | 47 |
| 0.5% DMSO | 48 | 65 | 73 | 64 | 66 | 63 | 27 |
| Control peptide | 68 | 62 | 61 | 68 | 66 | 65 | 29 |
| Bad (SEQ ID NO: 1) | 46 | 56 | 42 | 47 | 45 | 47 | 4 |
| Bad 2Ser/2Ala | 12 | 12 | 10 | 22 | 12 | 14 | 2 |
| (SEQ ID NO: 2) | | | | | | | |
| Bad Control 4 Ala | 46 | 45 | 26 | 48 | 38 | 41 | 20 |
| (SEQ ID NO: 4) | | | | | | | |

### EXAMPLE II

### Induction of Apoptosis Using Modified BAD Peptides with Poly-Arginine Cell Penetration Sequences

A. Apoptosis in CLL Cells by Modified BAD Peptides Including a Poly-Arginine Cell Penetration Sequence

The wild type and modified BAD peptides fused to poly-arginine cell penetration sequences shown in Table 5 were synthesized by standard solid phase synthesis. CLL cell viability assays were performed as described above except that cell permeable peptides SEQ ID NOS: 28, 6 and 29 and the control, non-permeable peptide SEQ ID NO: 3 (see Table 5) were added directed to CLL cells in culture without electroporation.

TABLE 5

Wild type and modified BAD peptides with
poly-arginine delivery agent sequences

| | |
|---|---|
| Peptide/SEQ ID NO: | Amino acid sequence |

| | |
|---|---|
| Wild type Bad poly(_{D}Arg) | NLWAAQRYGRELRRMSDEFVDSFKKC-Ahx*-(_{D}Arg)₈ |
| SEQ ID NO: 28 | |
| Bad 2Ser/Ala poly(_{D}Arg) | NLWAAQRYGRELRRMADEFVDAFKKC-Ahx-(_{D}Arg)₈ |
| SEQ ID NO: 6 | |
| Bad 3A control | NLWAAQRYGREARRMADEFVDAFKK |
| SEQ ID NO: 3 | |
| Bad 4A control poly(_{D}Arg) | NLWAAQRYGREARRMAAEFVDAFKKC-Ahx-(_{D}Arg)₈ |
| SEQ ID NO: 29 | |
| SEQ ID NO: 30 | AVPIAQK-Ahx-(_{D}Arg)₈ |

*Ahx is aminohexanoic acid

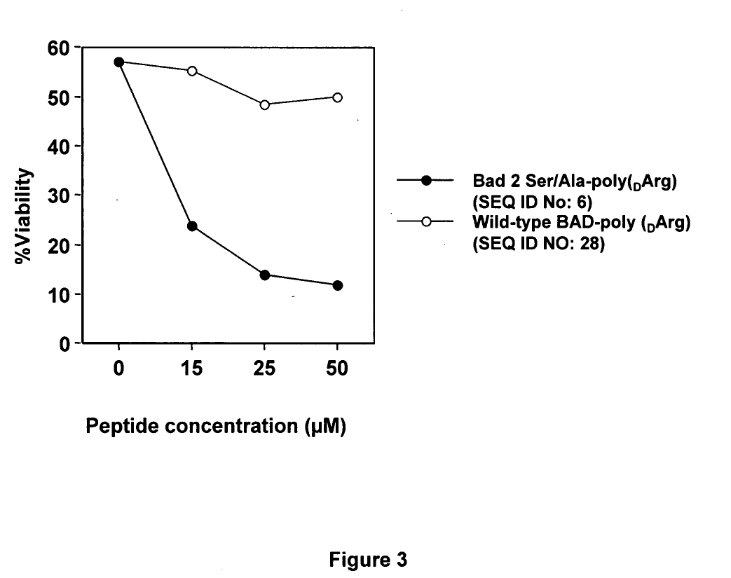

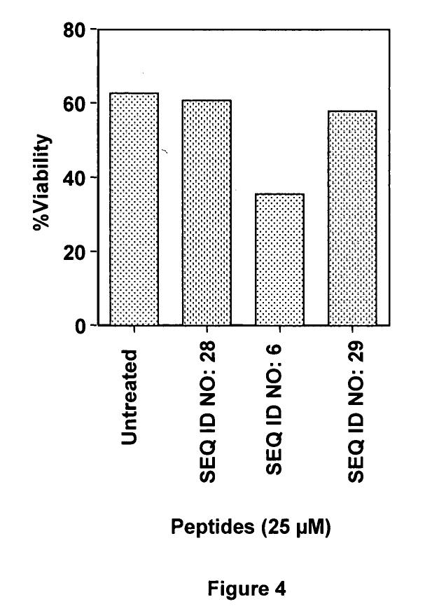

As shown in FIG. 3, addition of the modified BAD peptide, Bad 2Ser/Ala poly(_{D}Arg), SEQ ID NO: 6, resulted in a dose-dependent decrease in cell viability in patient TJK379 CLL cells. In particular, the percentage cell viability was dramatically reduced with as little as 15 μM peptide. In contrast, addition of wild type Bad poly(_{D}Arg) peptide SEQ ID NO: 28 had a negligible effect when added to CLL cells, even at a concentration of 50 μM. These results were corroborated in TJK197 patient cells. As shown in FIG. 4, cell viability was reduced from about 60% to less than 40% by the modified BAD peptide SEQ ID NO: 6, while no change in viability was observed with the wild type Bad poly(_{D}Arg) peptide SEQ ID NO: 28 or the Bad 4A control poly(_{D}Arg) peptide SEQ ID NO: 29.

These results demonstrate that a delivery agent such as a poly-arginine sequence can be sufficient for cellular uptake of modified BAD peptides and that the modified BAD peptide SEQ ID NO: 28 can dramatically reduce cell viability of cancer cells whereas a wild type BAD peptide has no effect.

B. Effect of Modified BAD Peptides on Bcl-2 Levels in CLL Cells.

TJK160 and TJK357 CLL patient cells were treated with the modified BAD peptide SEQ ID NO: 6 or with control peptides SEQ ID NO: 29 and SEQ ID NO: 30 essentially as described above. To determine the levels of Bcl-2 in each of the peptide treated CLL samples, a BD™ Cytometric Bead Assay (BD CBA; BD Biosciences) was used to quantitatively measure active caspase-3, cleaved PARP and Bcl-2 levels in a single sample. In this assay, soluble analytes are measured using amplified fluorescence detection and flow cytometry, with each bead in a CBA providing a capture surface for a specific protein and being analogous to an individually coated well in an ELISA plate. A single bead population with a distinct fluorescence intensity is coated with a capture antibody specific for active caspase-3 protein, cleaved PARP protein or Bcl-2 protein and subsequently resolved in the FL3 channel of a BD FACSTM brand flow cytometer. The capture bead, PE-conjugated detection antibody, and cell lysate standard or test samples are incubated together to form sandwich complexes and analyzed using BD CBA Analysis Software.

Cells were lysed in 1× CBA Cell Lysis Buffer supplemented with Protease Inhibitor Cocktail according to the manufacturer's protocol. Human active caspase-3, cleaved PARP and Bcl-2 lysate standards were reconstituted in deionized water at room temperature for 15 minutes. The standard was diluted by serial dilutions using assay diluent according to the manufacturer's protocol. Capture beads were vortexed before use, and 50 μl of capture beads were transferred to each assay tube. Standard dilutions and test samples were added to the appropriate sample tubes (50 μl/tube) and incubated at room temperature for one hour. After washing with 0.5 ml of Wash Buffer and centrifuging the samples, PE Detection Reagent was added (50 μl/tube), and the samples incubated at room temperature for one hour. All samples were subsequently washed with 0.5 ml of Wash Buffer and centrifuged. After addition of 300 μl of Wash Buffer, samples were analyzed by the BD FACScan or BD FACSCalibur. Samples were analyzed by the use of BD CellQuest Software, and data were acquired by the use of the BD CBA Software. A unit of active caspase-3, Bcl-2 or cleaved PARP is defined as the amount of active caspase-3, Bcl-2 or cleaved PARP in 0.1 μg of total protein from camptothecin-treated Jurkat cell lysate in a standard two hour assay protocol.

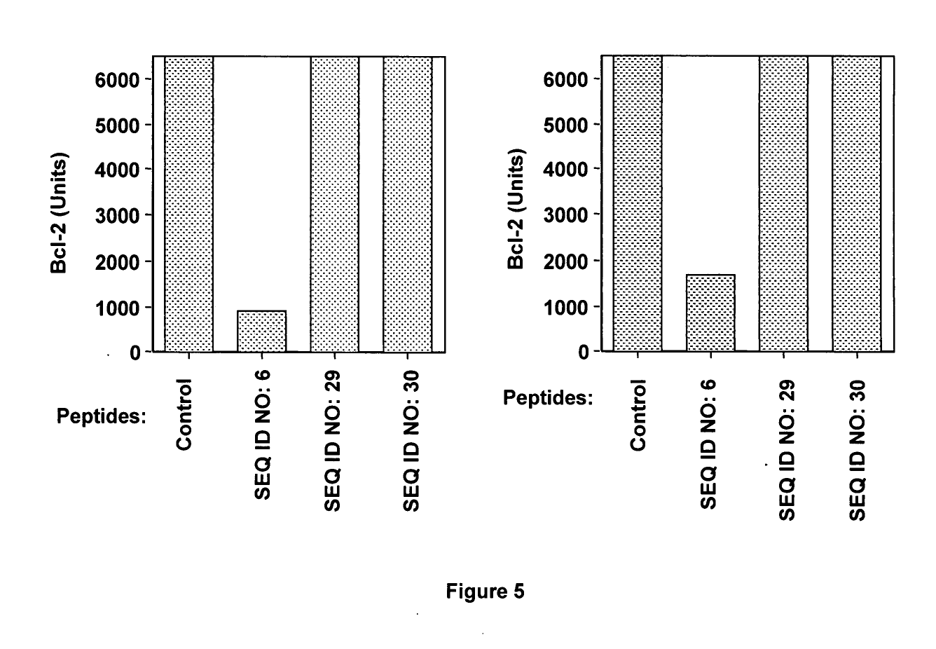

As shown in both the left and right panels of FIG. 5, treatment of CLL cells with the modified BAD peptide SEQ ID NO: 6 resulted in a significant reduction in intracellular levels of Bcl-2. This reduction was not observed with the control peptides SEQ ID NO: 29 or SEQ ID NO: 30. These results indicate that modified BAD peptides, but not wild type BAD peptides, can effect levels of the anti-apoptotic protein Bcl-2 in cancer cells such as leukemic cancer cells.

### EXAMPLE III

### Induction of Apoptosis Using Modified BAD Peptides Fused to Penetratin

The Bad 2Ser/Ala peptide (SEQ ID NO:2) was synthesized as a disulfide linked fusion peptide with penetratin-1 (SEQ ID NO: 27). The peptide, designated 2335, a BAD-BH3 peptide, has the sequence: acetyl-NLWAAQRYGRELRRMADEFVDAFKKC-CRQIKIWFQNRRMKWKK-amide (SEQ ID NO: 31). The C—C indicates the linkage of the Bad 2S/A peptide to the cell-penetrating peptide, penetratin, by a disulfide linkage. An enantiomer control peptide was designated 2336: acetyl-NLWAAQRYGRELRRMADEFVDAFKKC-CRQIKIWFQNRRMKWKK-amide (SEQ ID NO: 32). The 2336 peptide is an all D-amino acid peptide that does not bind Bcl-2 family Bcl-2/x1 and is an inactive control peptide. Both peptides are acetylated at the N-terminus and amidated at the C-terminus.

The peptides were tested in cell viability assays essentially as described above in Examples I and II. Experiments were carried out in 697-neo and 697-Bcl-2 cells. 697 cells are from a cell line of preB leukemic cells. 697-Bcl-2 cells have been engineered to express high levels of Bcl-2. AML-349 and AML-728 are myeloblasts (verified by FACS analysis via CD34-positivity) that were isolated from respective patients with acute myelogenous leukemia (AML).

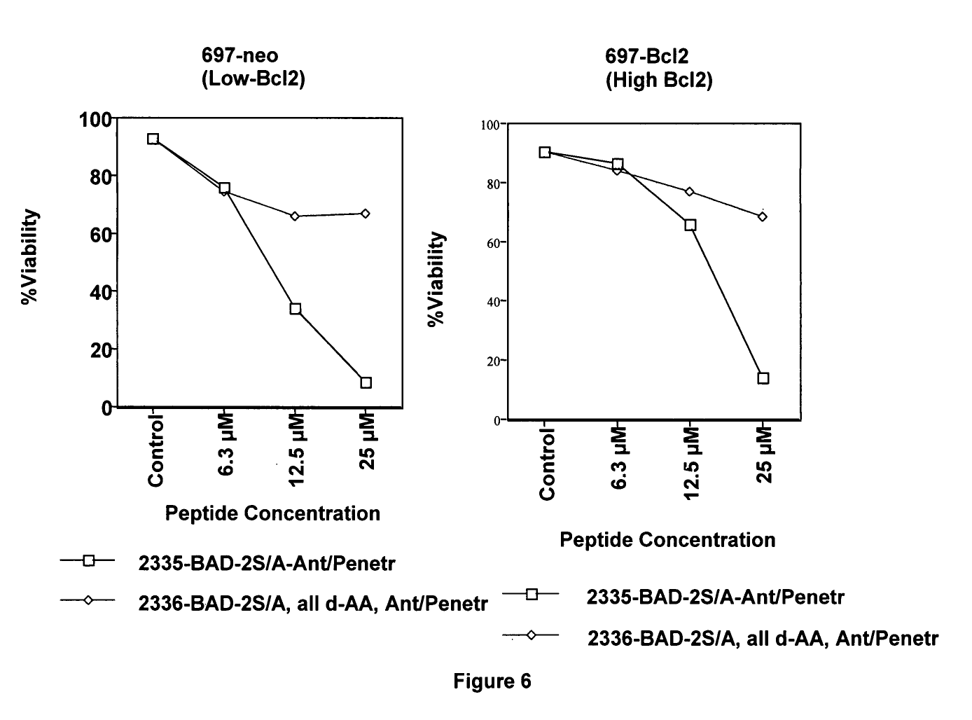

FIG. 6 shows that the 2335 peptide with Bad S/A disulfide linked to penetratin is more active in 697-neo cells with low Bcl-2 than a 2336 control peptide, which cannot bind to Bcl-2 (FIG. 6, left panel). Also, it takes more 2335 to kill 697 cells with higher levels of Bcl-2 (FIG. 6, right panel), indicating that Bcl-2 binds 2335 in cells to block its action, which can then be overcome by adding more 2335 peptide.

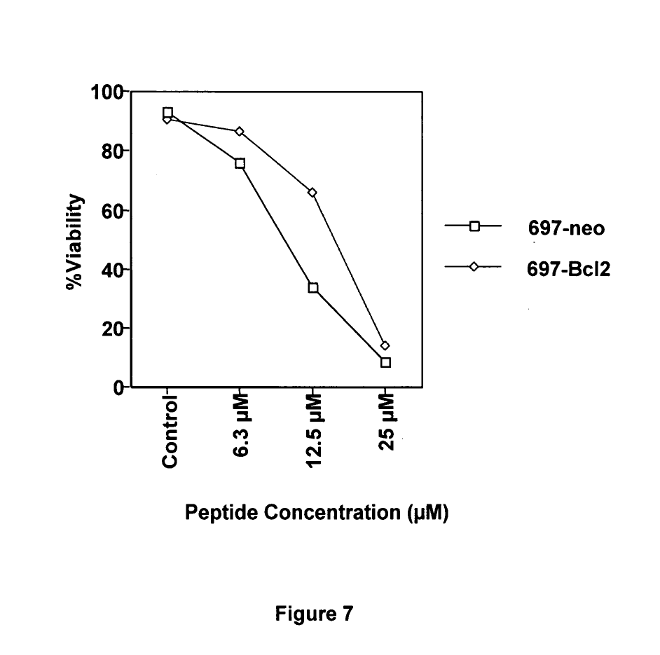

FIG. 7 plots the curves for the 2335 peptide from FIG. 6 on the same graph. The shift in the dose response curve can be seen in cells overexpressing Bcl-2 (697-Bcl2), indicating that the 2335 peptide induced apoptosis via antagonizing against Bcl-2 protein. FIG. 7 thus shows more directly how increasing Bcl-2 in cells can block its action, which can then be overcome by adding more 2335 peptide.

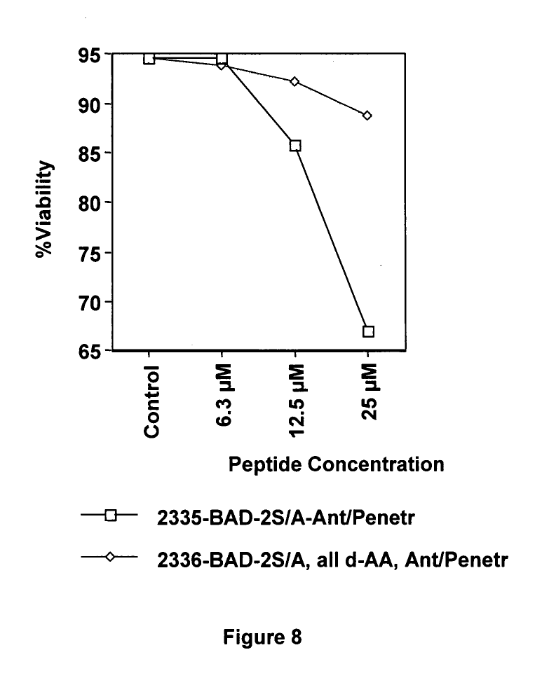

FIG. 8 shows that the 2335 peptide induced apoptosis in AML (acute myelogenous leukemia) cells isolated from a patient (AML-728), whereas the 2336 control peptide does not. Since the 2335 peptide binds Bcl-2 while the 2336 peptide does not, the experiment provides strong evidence that AML cells can be killed by targeting the 2335 peptide. This represents an important finding since it identifies a single agent target in leukemia cells (Bcl-2) that can be effectively targeted with the 2335 peptide.

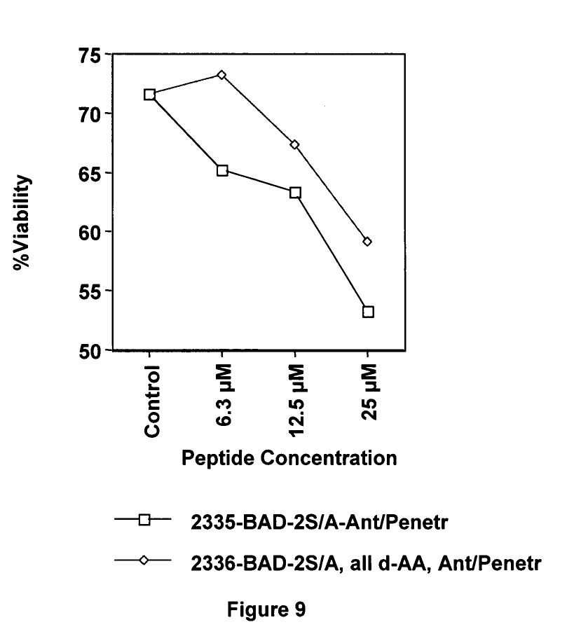

FIG. 9 shows that the 2335 peptide induced apoptosis in AML cells isolated from a second patient (AML-349) while the control peptide 2336 showed less killing.

The peptides fused to penetratin also exhibited advantageous properties of less non-specific toxicity than the Arg₈ fused peptides.

These results show that Bcl-2/x1 antagonists are useful therapeutic agents to overcome chemoresistance in AML.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the following claims.

What is claimed is:

1. A modified BAD peptide or peptidomimetic, comprising an amino acid sequence having at least 60% amino acid identity with SEQ ID NO: 1,
said modified BAD peptide or peptidomimetic having enhanced affinity for Bcl-2 as compared to wild type BAD peptide (SEQ ID NO: 1).

2. The modified BAD peptide or peptidomimetic of claim 1, comprising alanine at positions corresponding to residues 16 and 22 of SEQ ID NO: 1.

3. The modified BAD peptide or peptidomimetic of claim 1, comprising the amino acid sequence NLWAAQRYGRELRRMADEFVDAFKK (SEQ ID NO: 2) or a conservative variant or peptidomimetic thereof.

4. The modified BAD peptide or peptidomimetic of claim 3, consisting of the amino acid sequence SEQ ID NO: 2 or a peptidomimetic thereof.

5. The modified BAD peptide or peptidomimetic of claim 3, further comprising a penetratin (SEQ ID NO:27) fusion.

6. The modified BAD peptide or peptidomimetic of claim 5, comprising the amino acid sequence NLWAAQRYGRELRRMADEFVDAFKKC-CRQIKIWFQNRRMKWKK (SEQ ID NO:31).

7. The modified BAD peptide or peptidomimetic of claim 1, 2 or 3, which is a peptide.

8. A composition, comprising a modified BAD peptide or peptidomimetic and a delivery agent, said modified BAD peptide or peptidomimetic comprising an amino acid sequence having at least 60% amino acid identity with SEQ ID NO: 1, and having enhanced affinity for Bcl-2 as compared to wild type BAD peptide (SEQ ID NO: 1).

9. The composition of claim 8, wherein said modified BAD peptide or peptidomimetic comprises alanine at positions corresponding to residues 16 and 22 of SEQ ID NO: 1.

10. The composition of claim 8, wherein said modified BAD peptide or peptidomimetic comprises the amino acid sequence NLWAAQRYGRELRRMADEFVDAFKK (SEQ ID NO: 2) or a conservative variant or peptidomimetic thereof.

11. The composition of claim 10, wherein said modified BAD peptide or peptidomimetic consists of the amino acid sequence (SEQ ID NO: 2) or a peptidomimetic thereof.

12. The composition of claim 10, further comprising a penetratin (SEQ ID NO:27) fusion.

13. The composition of claim 12, comprising the amino acid sequence NLWAAQRYGRELRRMADEFVDAFKKC-CRQIKIWFQNRRMKWKK (SEQ ID NO:31).

14. The composition of claim 8, 9 or 10, wherein said modified BAD peptide or peptidomimetic is a peptide.

15. The composition of claim 8, wherein said delivery agent is covalently linked to said modified BAD peptide or peptidomimetic.

16. The composition of claim 8, which is a chimeric protein, peptide or peptidomimetic comprising said delivery agent operatively fused to said modified BAD peptide or peptidomimetic.

17. The composition of claim 8 or claim 16, wherein said delivery agent is a polycationic homopolymer.

18. The composition of claim 17, wherein said polycationic homopolymer is a polyarginine sequence.

19. The composition of claim 18, wherein said polyarginine sequence is (_{D}Arg)₈ (SEQ ID NO: 5).

20. The composition of claim 19, comprising the amino acid sequence NLWAAQRYGRELRRMADEFVDAFKKC-Ahx-(_{D}Arg) ₈ (SEQ ID NO: 6), wherein Ahx is aminohexanoic acid.

21. The composition of claim 8, wherein said delivery agent is non-covalently associated with said modified BAD peptide or peptidomimetic.

22. A method of treating leukemia in a patient, comprising administering to said patient a composition comprising a modified BAD peptide or peptidomimetic and a delivery agent, said modified BAD peptide or peptidomimetic comprising an amino acid sequence having at least 60% amino acid identity with SEQ ID NO: 1, and having enhanced affinity for Bcl-2 as compared to wild type BAD peptide (SEQ ID NO: 1).

23. The method of claim 22, wherein said leukemia is chronic lymphocytic leukemia.

24. The method of claim 22, wherein said modified BAD peptide or peptidomimetic comprises alanine at positions corresponding to residues 16 and 22 of SEQ ID NO: 1.

25. The method of claim 22, wherein said modified BAD peptide or peptidomimetic comprises the amino acid sequence NLWAAQRYGRELRRMADEFVDAFKK (SEQ ID NO: 2) or a conservative variant or peptidomimetic thereof.

26. The method of claim 22, wherein said modified BAD peptide or peptidomimetic consists of the amino acid sequence (SEQ ID NO: 2) or a peptidomimetic thereof.

27. The method of claim 25, wherein said modified BAD peptide or peptidomimetic further comprises a penetratin (SEQ ID NO:27) fusion.

28. The method of claim 27, wherein said penetratin fusion comprises the amino acid sequence NLWAAQRYGRELRRMADEFVDAFKKC-CRQIKIWFQNRRMKWKK (SEQ ID NO:31).

29. The method of claim 22, 23, 24 or 25, wherein said modified BAD peptide or peptidomimetic is a peptide.

30. The method of claim 22, wherein said delivery agent is covalently linked to said modified BAD peptide or peptidomimetic.

31. The method of claim 22, wherein said composition is a chimeric protein, peptide or peptidomimetic comprising said delivery agent operatively fused to said modified BAD peptide or peptidomimetic.

32. The method of claim 31, wherein said delivery agent is a polycationic homopolymer.

33. The method of claim 32, wherein said polycationic homopolymer is a polyarginine sequence.

34. The method of claim 33, wherein said polyarginine sequence is (_{D}Arg)₈ (SEQ ID NO: 5).

35. The method of claim 34, comprising the amino acid sequence NLWAAQRYGRELRRMADEFVDAFKKC-Ahx-(_{D}Arg)₈ (SEQ ID NO: 6), wherein Ahx is aminohexanoic acid.

36. The method of claim 22, wherein said delivery agent is non-covalently associated with said modified BAD peptide or peptidomimetic.


SEQUENCE LISTING (38 sequences)

SEQ ID NO: 1 - protein, 25 aa, Homo sapiens
NLWAAQRYGRELRRMSDEFVDSFKK

SEQ ID NO: 2 - protein, 25 aa, Artificial Sequence
  chemically synthesized
NLWAAQRYGRELRRMADEFVDAFKK

SEQ ID NO: 3 - protein, 25 aa, Artificial Sequence
  chemically synthesized
NLWAAQRYGREARRMADEFVDAFKK

SEQ ID NO: 4 - protein, 25 aa, Artificial Sequence
  chemically synthesized
NLWAAQRYGREARRMAAEFVDAFKK

SEQ ID NO: 5 - protein, 8 aa, Artificial Sequence
  chemically synthesized
RRRRRRRR

SEQ ID NO: 6 - protein, 35 aa, Artificial Sequence
  chemically synthesized
NLWAAQRYGRELRRMADEFVDAFKKCXRRRRRRRR

SEQ ID NO: 7 - protein, 25 aa, Artificial Sequence
  chemically synthesized
NLWAAQRYGRELRRMADEFVDSFKK

SEQ ID NO: 8 - protein, 25 aa, Artificial Sequence
  chemically synthesized
NLWAAQRYGRELRRMSDEFVDAFKK

SEQ ID NO: 9 - protein, 16 aa, Artificial Sequence
  chemically synthesized
AAVALLPAVLLALLAP

SEQ ID NO: 10 - protein, 12 aa, Artificial Sequence
  chemically synthesized
TPPKKKRKVEDP

SEQ ID NO: 11 - protein, 27 aa, Artificial Sequence
  chemically synthesized
GWTLNSAGYLLGKINLKALAALAKKIL

SEQ ID NO: 12 - protein, 11 aa, Artificial Sequence
  chemically synthesized
YGRKKRRQRRR

SEQ ID NO: 13 - protein, 16 aa, Artificial Sequence
  chemically synthesized
KKWKMRRNQFWIKIQR

SEQ ID NO: 14 - protein, 16 aa, Artificial Sequence
  chemically synthesized
RQIKIWFPNRRMKWKK

SEQ ID NO: 15 - protein, 16 aa, Artificial Sequence
  chemically synthesized
RQPKIWFPNRRMPWKK

SEQ ID NO: 16 - protein, 16 aa, Artificial Sequence
  chemically synthesized
RQIKIWFQNMRRKWKK

SEQ ID NO: 17 - protein, 16 aa, Artificial Sequence
  chemically synthesized
RQIRIWFQNRRMRWRR

SEQ ID NO: 18 - protein, 16 aa, Artificial Sequence
  chemically synthesized
RRWRRWWRRWWRRWRR

SEQ ID NO: 19 - protein, 4 aa, Artificial Sequence
  chemically synthesized
SCWK

SEQ ID NO: 20 - protein, 4 aa, Artificial Sequence
  chemically synthesized
LARL

SEQ ID NO: 21 - protein, 19 aa, Artificial Sequence
  chemically synthesized
KKKKKKKKKKKKKKKKRGD

SEQ ID NO: 22 - protein, 23 aa, Artificial Sequence
  chemically synthesized
ALKCWKKKKKKKKKKKKKKKKKK

SEQ ID NO: 23 - protein, 22 aa, Artificial Sequence
  chemically synthesized
DICWKKKKKKKKKKKKKKKKKK

SEQ ID NO: 24 - protein, 4 aa, Artificial Sequence
  chemically synthesized
PLAE

SEQ ID NO: 25 - protein, 5 aa, Artificial Sequence
  chemically synthesized
KPLAE

SEQ ID NO: 26 - protein, 27 aa, Artificial Sequence
  chemically synthesized
GALFLGFLGAAGSTMGAWSQPKSKRKV

SEQ ID NO: 27 - protein, 16 aa, Artificial Sequence
  chemically synthesized
RQIKIWFQNRRMKWKK

SEQ ID NO: 28 - protein, 35 aa, Artificial Sequence
  chemically synthesized
NLWAAQRYGRELRRMSDEFVDSFKKCXRRRRRRRR

SEQ ID NO: 29 - protein, 35 aa, Artificial Sequence
  chemically synthesized
NLWAAQRYGREARRMAAEFVDAFKKCXRRRRRRRR

SEQ ID NO: 30 - protein, 16 aa, Artificial Sequence
  chemically synthesized
AVPIAQKXRRRRRRRR

SEQ ID NO: 31 - protein, 43 aa, Artificial Sequence
  chemically synthesized
NLWAAQRYGRELRRMADEFVDAFKKCCRQIKIWFQNRRMKWKK

SEQ ID NO: 32 - protein, 43 aa, Artificial Sequence; identical to SEQ ID NO: 31
  chemically synthesized

SEQ ID NO: 33 - protein, 30 aa, Artificial Sequence
  chemically synthesized
XALXNLWAAQRYGRELRRMADEFVDAFKKC

SEQ ID NO: 34 - protein, 30 aa, Artificial Sequence
  chemically synthesized
XAAXNLWAAQRYGRELRRMADEFVDAFKKC

SEQ ID NO: 35 - protein, 33 aa, Artificial Sequence
  chemically synthesized
XAAXRYGRELRRMADEFVDAFKKCXRRRRRRRR

SEQ ID NO: 36 - protein, 24 aa, Artificial Sequence
  chemically synthesized
XALXRYGRELRRMADEFVDAFKKC

SEQ ID NO: 37 - protein, 24 aa, Artificial Sequence
  chemically synthesized
XAAXRYGRELRRMADEFVDAFKKC

SEQ ID NO: 38 - protein, 39 aa, Artificial Sequence
  chemically synthesized
XALXNLWAAQRYGRELRRMADEFVDAFKKCXRRRRRRRR